(19) United States
(12) Patent Application Publication
(10) Pub. No.: US 2022/0152414 A1
(43) Pub. Date: May 19, 2022

(54) SYSTEMS AND METHODS FOR CATHETER BASED LIGHT DELIVERY

(71) Applicants: THE GENERAL HOSPITAL CORPORATION, Boston, MA (US); BOSTON SCIENTIFIC CORPORATION, Marlborough, MA (US)

(72) Inventors: William G. Austen, Weston, MA (US); Robert Redmond, Newton Centre, MA (US); Michael McCormack, Medford, MA (US); Matt Hart, Boston, MA (US); Martha Wittrock, Boston, MA (US); Lisa Shoemaker, Boston, MA (US); Tom Jancaric, Boston, MA (US)

(21) Appl. No.: 17/441,200

(22) PCT Filed: Mar. 23, 2020
(86) PCT No.: PCT/US2020/024231

§ 371 Date: Sep. 20, 2021

Related U.S. Application Data
(60) Provisional application No. 62/821,799, filed on Mar. 21, 2019.
(60) Provisional application No. 62/821,803, filed on Mar. 21, 2019.
(60) Provisional application No. 62/821,806, filed on Mar. 21, 2019.

(51) Int. Cl. A61N 5/06; A61B 1/018

(52) U.S. Cl. CPC A61N 5/0603; A61B 2018/00285; A61N 5/062; A61B 1/018

(57) ABSTRACT

Systems and methods are provided for performing a treatment of a lesion within a subject using an endoscopic device. The endoscopic device comprises an endoscope and a catheter. The endoscope is configured to be inserted into the subject and includes a working channel. The catheter is configured to pass through the working channel of the endoscope. The catheter further comprises a sheath, an expandable device, and a light source. The sheath has a lumen and the expandable device may include an inflatable balloon. The light source is configured to be controllable through the lumen of the sheath. The light source is configured to selectively apply light to the lesion within the subject when the endoscope is inserted into the subject.

### CROSS-REFERENCE TO RELATED APPLICATIONS

This application is based on, claims priority to, and incorporates herein by reference in its entirety, each of U.S. Provisional Application Ser. Nos. 62/821,799; 62/821,803; and 62/821,806, each of which was filed on Mar. 21, 2019, and entitled “SYSTEMS AND METHODS FOR CATHETER BASED LIGHT DELIVERY.”

### BACKGROUND OF THE INVENTION

Various surgical procedures for removing growths on a patient or subject (e.g., cysts, tumors, neoplasms, etc.) may result in internal or external lesions left in the surrounding tissue of the patient or subject. In the case of procedures resulting in internal lesions, it may be particularly important to avoid post-operative bleeding, perforation, or stricture formation.

For example, in some instances, endoscopic mucosal resection (EMR) may be used to treat esophageal cancer. Similarly, endoscopic submucosal dissection (ESD) may be used to remove gastrointestinal tumors. In either of these procedures, treatment of the resulting lesion left in the surrounding tissue may be desired or necessary in preventing postoperative bleeding, perforation, and stricture formation.

However, present treatment methods may be time and/or cost prohibitive, and in some instances, may not provide adequate treatment to the lesion.

### SUMMARY

The present disclosure overcomes the above and other drawbacks by providing systems and methods for treating a lesion within a subject using a system designed for targeted lesion treatment.

In accordance with one aspect of the disclosure, a system is provided for delivering treatment to a lesion within a subject using an endoscopic device. The endoscopic device comprises an endoscope and a catheter. The endoscope is configured to be inserted into the subject and includes a working channel. The catheter is configured to pass through the working channel of the endoscope. The catheter further comprises a sheath, an expandable device, and a light source. The sheath has a lumen and light source is configured to be controllable through the lumen of the sheath. The light source is configured to selectively apply light to the lesion within the subject when the endoscope is inserted into the subject.

In accordance with another aspect of the disclosure, a method is provided for treating a lesion within a subject using an endoscopic device including an endoscope and a catheter. The endoscope includes a working channel and the catheter being configured to pass through the working channel of the endoscope and comprising an inflatable balloon and a light source. The method includes identifying the lesion within the subject, applying a photosensitizer to the lesion within the subject, inserting the endoscope into the subject, such that a distal end of the endoscope is proximate the lesion, and advancing the catheter through the working channel, into the subject, proximate the lesion. The method also includes inflating the inflatable balloon through the catheter within the subject and applying light to the photosensitizer using the light source to treat the lesion.

In accordance with yet another aspect of the disclosure, an endoscopic device is provided for treating a lesion within a subject. The endoscopic device includes an endoscope configured to be inserted into the subject, the endoscope including a working channel and a catheter configured to pass through the working channel of the endoscope. The catheter includes sheath having a lumen, an expandable device configured to move between a collapsed position configured to travel through the lumen and an expanded positon to engage the subject when the catheter is positioned within the subject, and a light source controllable through lumen of the sheath and extending into the expandable device. The expandable device is configured to engage the subject in the expanded positon to positon the light source to selectively apply light to the lesion within the subject when the endoscope is inserted into the subject.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic illustration of an endoscopic system in accordance with the present disclosure.

FIG. 2 is an elevational front view of an endoscopic device of the endoscopic system of FIG. 1.

FIG. 3 is a side view of the endoscopic device of FIG. 2, shown with a catheter advanced out of the endoscopic device in a collapsed position.

FIG. 4 is a side view of the endoscopic device of FIG. 3, shown with the catheter in an expanded position.

FIG. 5A is a side view of a sheath of the catheter of FIG. 4.

FIG. 5B is an elevational front view of the sheath of FIG. 5A.

FIG. 6 is a perspective view of an actuation tube and a guide wire tube in accordance with the present disclosure.

FIG. 7 is a schematic view of the endoscopic device of FIG. 2, shown inserted into an internal passageway within a subject.

FIG. 8 is a process flowchart illustrating an exemplary method of use of the endoscopic system of FIG. 1.

FIG. 9 is a side view of the endoscopic device of FIG. 2, shown including advancement indicators on the catheter.

FIG. 10A is a side view of an expandable device of the catheter of FIG. 4, shown including a plurality of microholes.

FIG. 10B is a schematic view of a single-file line pattern of microholes applied to the expandable device of FIG. 4.

FIG. 100 is a schematic view of a staggered line pattern of microholes applied to the expandable device of FIG. 4.

FIG. 11 is a schematic view of another catheter in accordance with the present disclosure.

FIG. 12 is a schematic view of another catheter in accordance with the present disclosure.

FIG. 13 is a schematic view of another catheter in accordance with the present disclosure, shown with the catheter in a collapsed position.

FIG. 14 is a cross-sectional view of the catheter of FIG. 13, taken along line 14-14.

FIG. 15 is a schematic view of the catheter of FIG. 13, shown in an expanded position.

FIG. 16 is a schematic view of another catheter in accordance with the present disclosure, shown with the catheter in a compressed position.

FIG. 17 is a schematic view of the catheter of FIG. 16, shown in an expanded view.

### DETAILED DESCRIPTION OF THE INVENTION

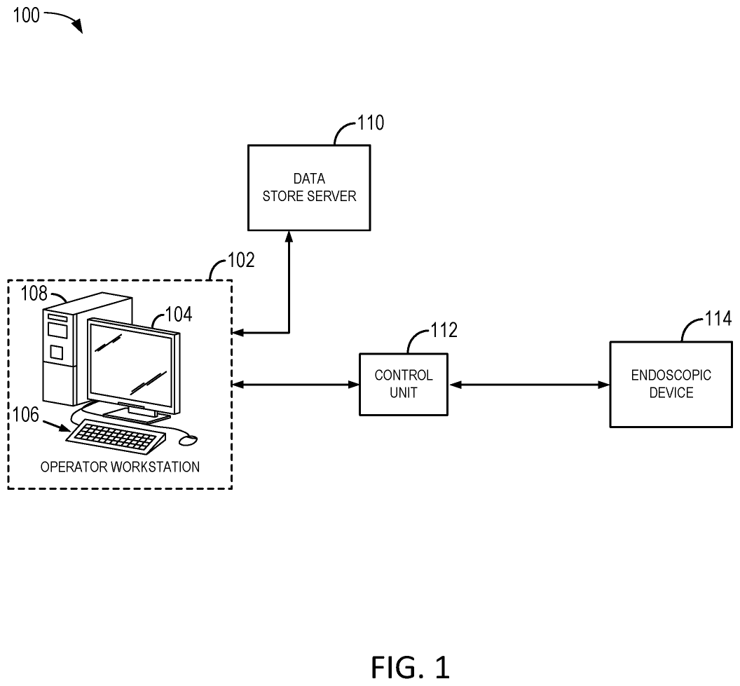

Referring to FIG. 1, an exemplary endoscopic system 100 is provided that may be used to treat internal lesions within a subject. As illustrated, the endoscopic system 100 may include an operator workstation 102 that may include a display 104, one or more input devices 106 (e.g., a keyboard, a mouse, etc.), and a processor 108. The processor 108 may include a commercially available programmable machine running a commercially available operating system. The operator workstation 102 provides an operator interface that facilitates entering various treatment parameters that will be described below into the endoscopic system 100. The operator workstation 102 may be coupled to a data store server 110. The operator workstation 102 and the data store server 110 may be connected via a wired or wireless network connection. In some instances, the operator workstation 102 may be configured to acquire various predetermined treatment parameters from the data store server 110 based on input provided to the operator workstation 102.

The endoscopic system 100 may further include a control unit 112 and an endoscopic device 114. The control unit 112 may be in communication with both the operator workstation 102 and the endoscopic device 114. Accordingly, the control unit 112 may be configured to receive treatment parameters from the operator workstation 102, and to subsequently apply those treatment parameters to the endoscopic device 114.

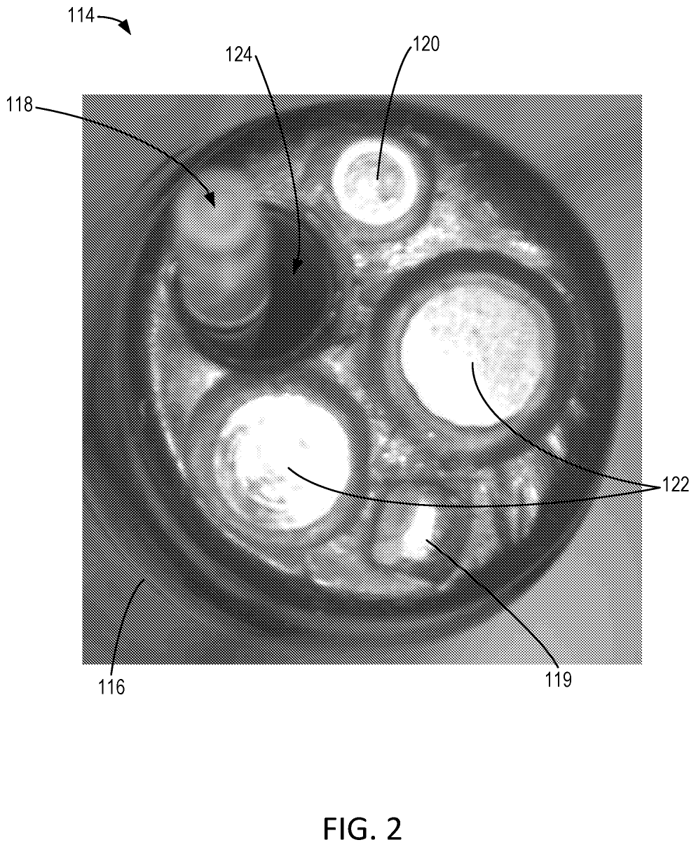

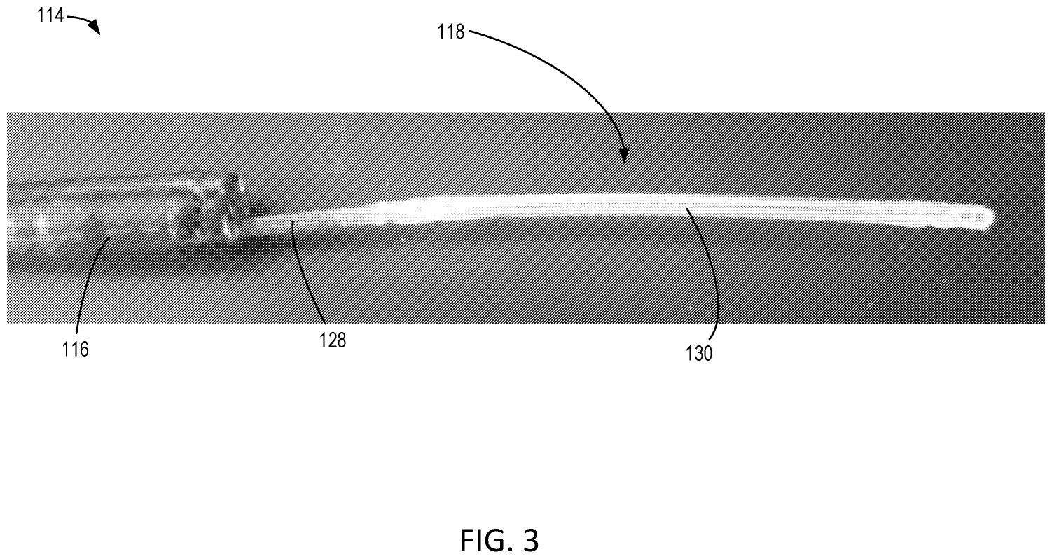

Referring now to FIGS. 2 and 3, the endoscopic device 114 comprises an endoscope 116 and a catheter 118. The endoscope 116 may be configured to be inserted into an internal space within the subject. The endoscope 116 may include an endoscope light 119, a lens 120, guide wires 122, and a working channel 124.

The endoscope light 119 may be configured to apply visible treatment light to the internal space within the subject during treatment. In some instances, the lens 120 may comprise a physical lens for conveying an image to an eyepiece to be used by a healthcare professional during treatment. Additional or alternative, the lens 120 may comprise a camera lens configured to convey a real-time image of the internal space of the subject to a screen (not shown) to be used by the healthcare professional during treatment. The guide wires 122 may be used to control the direction of the endoscope during insertion into the internal space of the subject. Accordingly, during treatment, the endoscope light 119, the lens 120, and the guide wires 122 may be collectively used by the healthcare professional to aid in directing and placement of the endoscope 116 within the subject.

As illustrated in FIG. 2, the working channel 124 may be configured to receive the catheter 118. As such, the catheter 118 may be configured to be passed through the working channel 124. Specifically, as will be described below, the catheter 118 may be selectively moved between an expanded position and a collapsed position. As shown in FIG. 2, in the collapsed position, every component of the catheter 118 is sized to pass through the working channel 124 of the endoscope 116.

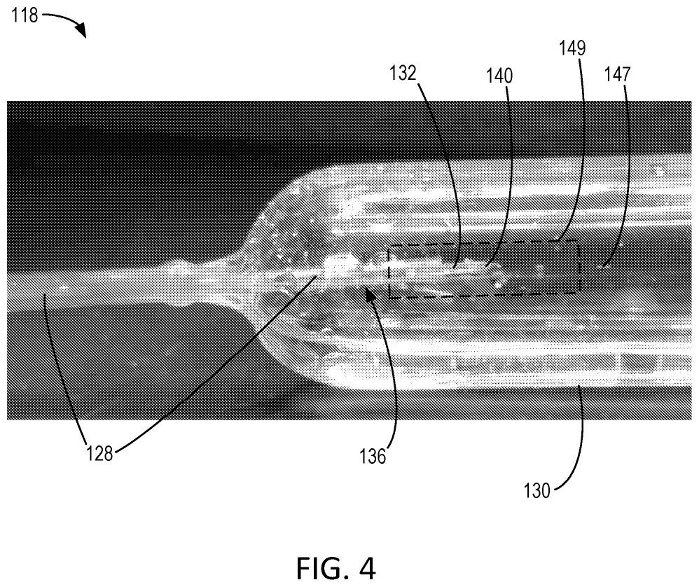

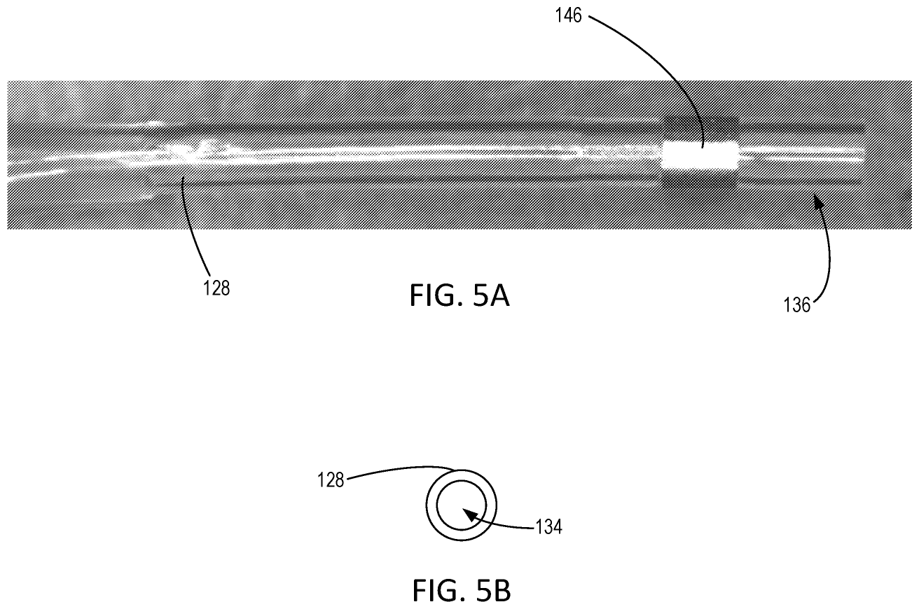

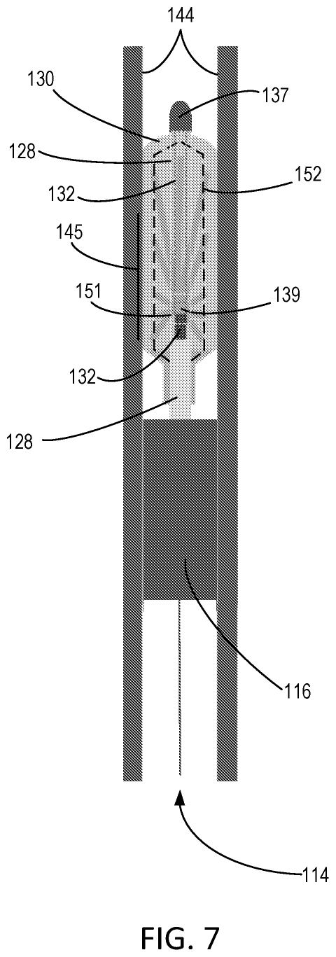

Referring now to FIGS. 3 and 4, the catheter 118 includes a sheath 128, an expandable device 130, and a light source 132 (shown in FIG. 4). The sheath 128 comprises a hollow tube having a lumen 134 (shown in FIG. 5B). The sheath 128 may comprise a polymeric material, such as, for example, nylons, urethanes, polyesters, or any other suitable polymeric material. In some instances, the sheath 128 may extend partially into the expandable device 130. For example, in some instances, the sheath 128 may extend into the expandable device 130 such that a distal end 136 of the sheath 128 is disposed at approximately the axial midpoint of the expandable device 130. In some other instances, the sheath 128 may extend fully through the expandable device 130, as shown in FIG. 7.

As illustrated between FIGS. 3 and 4, the expandable device 130 may be an inflatable balloon configured to be inflated through the lumen 134 of the sheath 128. Accordingly, the expandable device 130 may be movable between a collapsed or deflated position (as shown in FIG. 3) and an expanded or inflated position (shown in FIG. 4). In the collapsed position, the inflatable balloon may be sized to move through the lumen 134 of the catheter 118. In the expanded position, the inflatable balloon may be configured to engage the subject after being deployed from the working channel 124 (shown in FIG. 7). The expandable device 130 may comprise a polymeric material, such as, for example Polyethylene terephthalate (PET), nylon, or any other suitable polymeric material.

The expandable device 130 may be sized according to the intended procedure to be done using the catheter 118. For example, in some instances, when the catheter 118 is to be used within the esophagus of a subject, the expandable device 130 may have a maximum expanded width or diameter of 25 mm. In some other instances, the expandable device 130 may have a maximum expanded width or diameter between 14 mm and 25 mm. In some instances, based on tissue anatomy of the patient or subject, the expandable device 130 may have a maximum expanded width up to 75 mm.

Further, in some instances, the expandable device 130 may have an axial length that is approximately one and a half times the length of the lesion to be treated. In some other instances, the expandable device 130 may have an axial length that is between one and three times the length of the lesion to be treated. In yet some other instances, the expandable device 130 may have an axial length that is at least as long as the lesion to be treated. In still some other instances, the expandable device 130 may have an axial length that is less than the length of the lesion to be treated, in which case multiple treatments may be used to treat the entire lesion. For example, in some instances, the expandable device 130 may have an axial length of approximately 4 cm. In some other instances, the expandable device 130 may have an axial length between approximately 2 cm and 6 cm.

The expandable device 130 may be coupled to the sheath 128, proximate the distal end 136 of the sheath 128. In some instances, the expandable device 130 may be coupled to the sheath 128 using an adhesive, polymeric welding, or any other suitable coupling method. In some instances the expandable device 130 may be coupled to the sheath 128 using a UV adhesive. The expandable device 130 and/or sheath 128 may additionally be sealed by or coupled to an end cap 137 (shown in FIG. 7) at a distal end 138 of the catheter 118. The expandable device 130 and/or sheath 128 may similarly be coupled to the end cap 137 using an adhesive, polymeric welding, or any other suitable coupling method.

Referring again to FIG. 4, in some instances, the light source 132 may be configured to be passed through the lumen 134 of the sheath 128. The light source 132 may be fed through the lumen 134 such that the light source 132 protrudes from the sheath 128 a predetermined distance to treat a lesion within a subject, as will be described below. In some instances, when the sheath 128 extends throughout the expandable device 130, the light source 132 may also extend throughout the expandable device 130 to the distal end of the sheath 128. In these instances, the light source 132 may be coupled to the sheath 128 at a distal end.

As illustrated, the light source 132 may comprise a fiber optic cable. The light source 132 may further comprise a guide wire coupled to or enveloping the fiber optic cable to aid in accurate placement of the light source 132 within the subject. The guide wire may be configured to arrange the light source 132 in a desired position within the inflatable balloon.

In some instances, a distal end 140 of the fiber optic cable may present an opening forming an angle relative to an optical path through the fiber optic cable, to facilitate accurate light emission from the light source 132. For example, in some instances, the fiber optic cable may comprise a 700 micron side-firing fiber optic cable. In some other instances, the fiber optic cable may comprise between a 200 micron and a 900 micron side-firing fiber optic cable. In other instances the optical cable may be an end-firing optic cable with an end, for example, that is normal to the cable axis (i.e., an end-firing cable configuration). Further still, an end-firing optic cable may be utilized, where the end is cut at a desired angle, such as, for example, at 180 degrees.

In some instances, the distal end 140 of the light source 132 may present an opening forming an angle of 35-50 degrees relative to the optical path through the fiber optic cable. In some instances, the distal end 140 of the fiber optic cable may instead comprise a rounded tip configured to disperse light evenly in 360 degrees. The tip is manipulated to alter the beam profile. A rounded tip is used to shape the beam to the size of the lesion and the angle is used to direct the beam towards the lesion. The distal end 140 may additionally be covered by a clear cover or cap (not shown) to protect the light source 132.

In yet some other instances, when the light source 132 extends entirely through the expandable device 130 (as illustrated by the dotted line in FIG. 7), the light source 132 may be cut at a light emission point 139 (also shown in FIG. 7), at a predetermined location within the expandable device 130, from which the light may be selectively emitted. In these instances, with the sheath 128 also extending entirely through the expandable device 130 (as illustrated by the dotted line in FIG. 7), the sheath 128 may have a window 151 cut therein to permit light to be emitted therethrough.

It will be appreciated that, although the emission point 139 and window 151 are illustrated near a proximal end of the expandable device 130, in some instances, the emission point 139 and window may be disposed elsewhere within the expandable device 130. For example, in some instances, the emission point 139 and window 151 may be disposed approximately at an axial midpoint of the expandable device 130. In some other instances, the emission point 139 and window 151 may be disposed proximate a distal end of the expandable device 130.

In some instances, the light source 132 may comprise a laser light passed through the fiber optic cable. In other instances, the light source 132 may comprise various other types of suitable light sources, such as, for example, a light emitting diode (LED), lamp or any other suitable light sources.

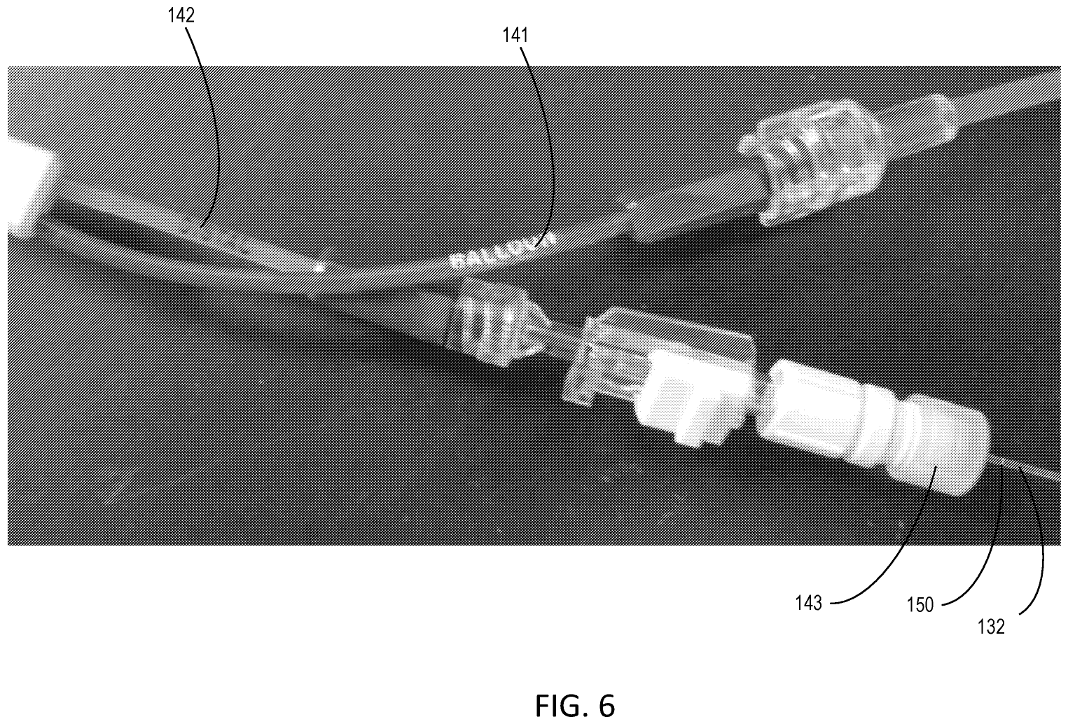

Referring now to FIG. 6, the sheath 128 may be operably connected to an actuation tube 141 and a guide wire tube 142. The actuation tube 141 may be configured to selectively expand or collapse the expandable device 130. For example, in the instances where the expandable device 130 is an inflatable balloon, the actuation tube 141 may be configured to provide a fluid (i.e., a gas ora liquid) into and through the sheath 128, into the inflatable balloon. In some instances, the fluid may be used to cool the fiber optic cable or other suitable light source 132. In these instances, the fluid provided into and through the sheath 128 and into the inflatable balloon may be recirculated through the sheath 128 and the inflatable balloon to aid in the cooling process. To further aide in cooling, this fluid may be recirculated through the balloon and catheter in a cooling loop. The guide wire tube 142 may comprise a hollow tube configured to receive the guide wires 122 and the light source 132, each of which may then be passed into the sheath 128. The guide wire tube 142 may further comprise a seal 143 configured to provide a seal around the light source 132 during use. In some instances, the seal 143 may be a Tuohy Borst adaptor. In some other instances the seal 143 may comprise other various other types of suitable sealing apparatuses.

Referring now to FIG. 7, during treatment, the endoscope 116 may be inserted into an internal passageway 144 (e.g., the esophagus, the colon, the intestinal tract, etc.) of a subject. The endoscope 116 may further be inserted such that the endoscope 116 is disposed proximate a lesion 145 within the internal passageway 144.

In some instances, to ensure that the light source 132 is centered within the expandable device 130, and thereby within the internal passageway 144, the catheter 118 may further include a secondary expandable device 152. In some instances, the secondary expandable device 152 may comprise a secondary inflatable balloon disposed within the expandable device 130. In some other instances, the secondary expandable device 152 may comprise a selectively expandable nitinol tube within the expandable device 130. In either case, the secondary expandable device 152 may be configured to receive the light source 132 therein. In some instances, the secondary expandable device 152 and the expandable device 130 may be configured to operate in concert to deliver a desired dispersion of light from the light source 132. In these instances, both the sheath 128 and the light source 132 may extend axially throughout both the expandable device 130 and the secondary expandable device 152 to further ensure that the light source 132 is centered within the internal passageway 144. In some instances, the secondary expandable device 152 may be covered with a silicon sheath or second balloon to provide structure and support via an initial inflation ahead of the dye being deployed.

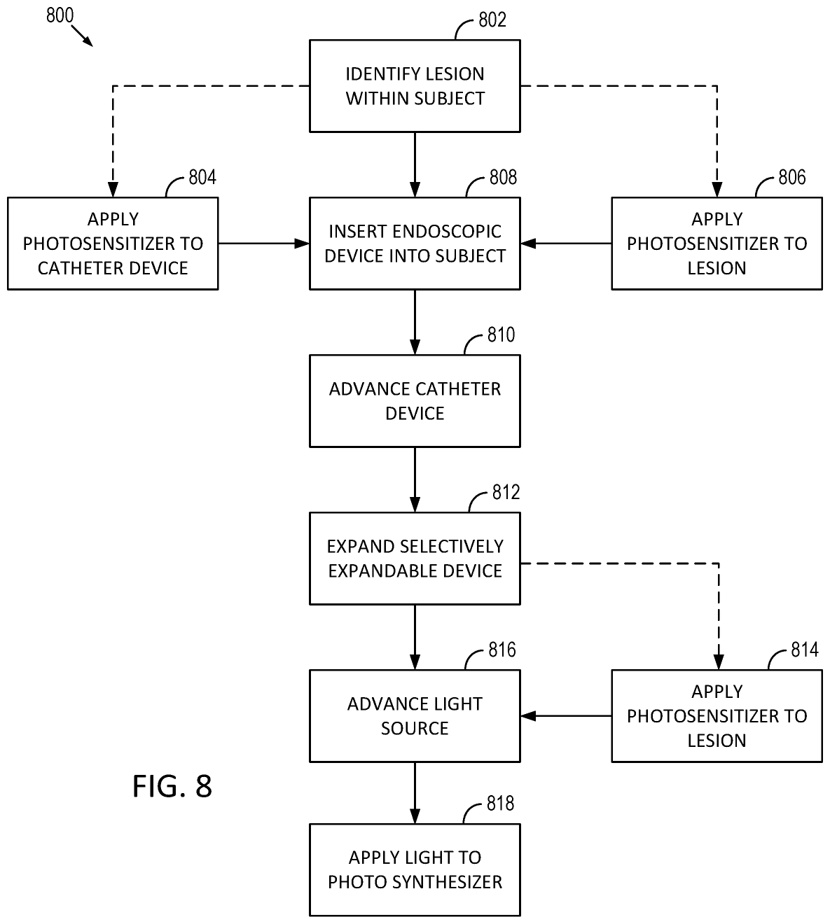

FIG. 8 provides an exemplary method of use 800 of the endoscopic device 114 during treatment. For example, in some instances, the method of use 800 may be used for treating a lesion in the tissue of a subject after undergoing endoscopic mucosal resection/endoscopic sub-mucosal dissection (EMR/ESD). It will be appreciated that the systems and devices described herein may be used for treating lesions resulting from various other procedures, and the following method of use is provided as a non-limiting example.

As shown, at step 802, a healthcare professional may first identify the lesion 145 within the internal passageway. This identification may be done using various imagining techniques, such as, for example, MRI, CT, ultrasound x-ray, endoscopic visualization, or any other suitable imaging technique.

In some instances, before or after the identification of the lesion 145 at step 802, a photosensitizer may be applied to the catheter 118, at step 804, to be subsequently applied to the lesion 145. The photosensitizer may, for example, be applied to an external surface of the expandable device 130. The photosensitizer may be configured to treat the lesion 145 when light is applied to the photosensitizer by the light source 132. For example, in some instances, the photosensitizer may comprise Rose Bengal. In other instances, the photosensitizer may comprise Rose Bengal (“RB”), erythrosine, riboflavin, methylene blue (MB), Toluidine Blue, Methyl Red, Janus Green B, Rhodamine B base, Nile Blue A, Nile Red, Celestine Blue, Remazol Brilliant Blue R, riboflavin-5-phosphate (R-5-P), N-hydroxypyridine-2-(I H)-thione (N-HTP) or photoactive derivatives thereof. In other instances, the photosensitizer may comprise xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, or anthroquinone dyes.

In some other instances, after the identification of the lesion 145 at step 802, the photosensitizer may be applied directly to the lesion 145. In these instances, the photosensitizer may be sprayed onto the lesion 145, rubbed onto the lesion 145 using a sponge-tipped device configured to be passed through the working channel 124 of the endoscope 116, applied using an injection needle, applied using a gel or film, or any other suitable method for applying the photosensitizer to the lesion 145. In yet some other instances, the photosensitizer may be applied to the lesion 145 through the expandable device 130, as will be described below.

After the lesion has been identified at step 802, and in some cases the photosensitizer has been applied to either the catheter 118 at step 804 or directly to the lesion 145 at step 806, the endoscopic device 114 may be inserted into the internal passageway 144 of the subject, at step 808. While inserting the endoscopic device 114 into the internal passageway 144, the endoscope light 119, the lens 120, and the guide wires 122 may be collectively used by the healthcare professional to ensure the endoscope 116 is placed proximate the lesion 145.

With the endoscopic device 114 disposed proximate the lesion 145, the catheter 118 may be advanced through the working channel 124 of the endoscope 116, at step 810. In some instances, to ease advancement of the catheter 118 through the working channel 124, a lubricant may be used on the outer surface of the catheter 118. For example, in some instances, the lubricant may be an MDX silicone lubricant. In either case, the distal end 138 of the catheter 118 may be advanced out of the endoscope 116, proximate the lesion 145.

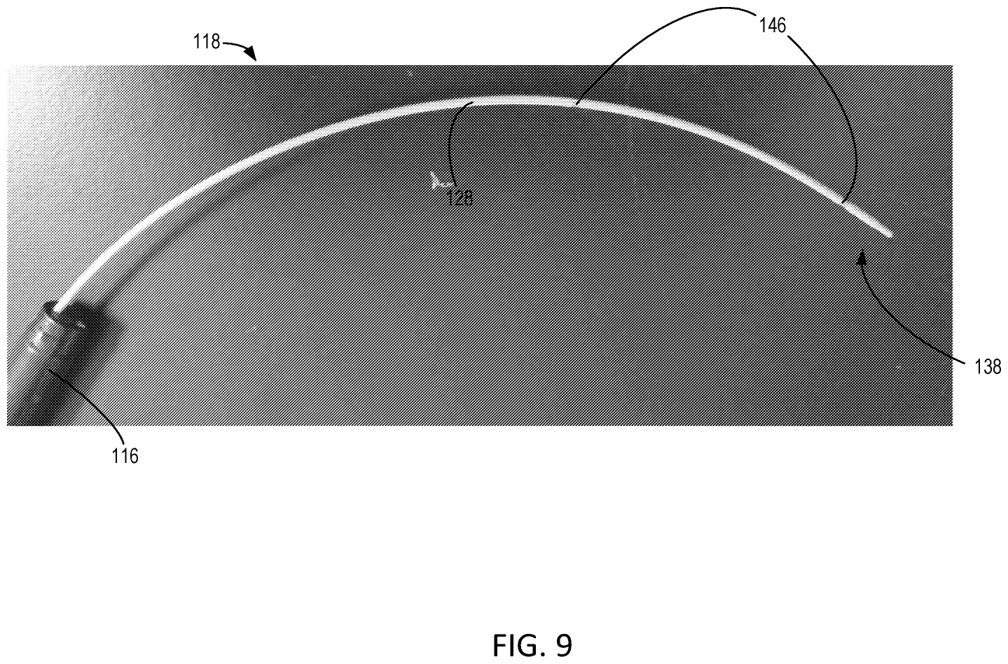

In some instances, the sheath 128 of the catheter 118 may include advancement indicators 146 (shown in FIG. 9). In some instances, the advancement indicators 146 may be colored or etched markings that may be viewed by the healthcare professional through the lens 120 during advancement of the catheter 118. In some instances, the advancement indicators 146 may comprise radiopaque bands that are friction fit over the sheath 128. The advancement indicators 146 may thus provide a measure of movement for the healthcare professional during advancement of the catheter 118 to ensure accurate placement of the catheter 118 proximate the lesion 145 within the internal passageway 144 of the subject.

After advancing the catheter 118 through the working channel 124 within the internal passageway 144 of the subject, the healthcare professional may then expand the expandable device 130 and/or the secondary expandable device 152, at step 812. In the instances where the expandable device 130 and the secondary expandable device 152 are inflatable balloons, the expandable device 130 and/or the secondary expandable device 152 may be expanded by supplying a pressurized fluid to the corresponding inflatable balloon through the lumen 134 of the sheath 128. In some instances, the pressurized fluid may comprise an inert gas or air. In some other instances, the pressurized fluid may comprise water, a lipid solution, a mixture of a scattering solution designed to distribute the light and water, or any other suitable dispersing solution. One non-limiting example of a scattering solution is intralipid, titanium dioxide, or other scattering medium. In some instances, when a mixture of intralipid and water is used, the fluid may be a 1:10 mix of intralipid to water. In some other instances, the fluid may be a 1:20 mix of intralipid to water. In other examples, the mixture may include 0.25-2% intralipid (scattering fat/water emulsion) or a TiO2.

As such, an outer surface of the inflatable balloon or expandable device 130 may contact an inner surface of the internal passageway 144 of the subject. This contact may provide an opening force on the inner surface of the internal passageway 144, such that the inflatable balloon is configured to selectively expand the internal passageway 144 of the subject. In some instances, the opening force provided to the inner surface of the internal passageway 144 may be controlled by supplying a predetermined volume of fluid to at least one of the inflatable balloons (i.e., the expandable device 130 and/or the secondary expandable device 152). In other instances, the opening force may be controlled using various pressure sensors to control the pressure of the fluid delivered to the at least one of the inflatable balloons.

In the instances where the photosensitizer has been applied to the external surface of the expandable device 130 at step 804, the contact between the expandable device 130 and the internal passageway 144 may additionally serve to apply the photosensitizer to the lesion 145.

In the instances where the photosensitizer has not been applied to the catheter 118 at step 804 or directly to the lesion at step 806, after expanding the expandable device 130 at step 812, the healthcare professional may then apply photosensitizer to the lesion 145 through the expandable device 130, at step 814.

In these instances, the expandable device 130 may include a plurality of microholes 148. In some instances, the plurality of microholes 148 may be ablated in the expandable device 130 using an ablative laser source. The plurality of microholes 148 may be arranged along a portion of the expandable device 130 that is configured to engage the subject when the expandable device 130 is in the expanded position.

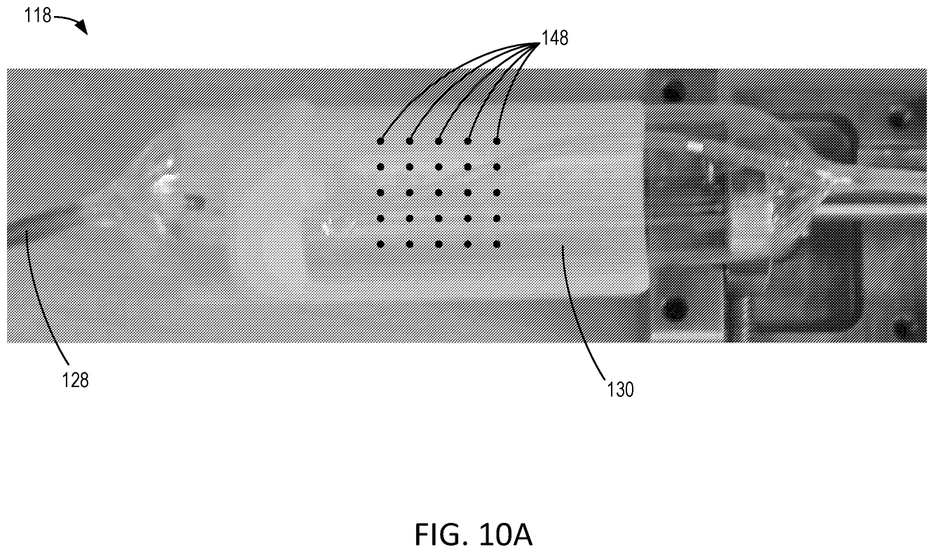

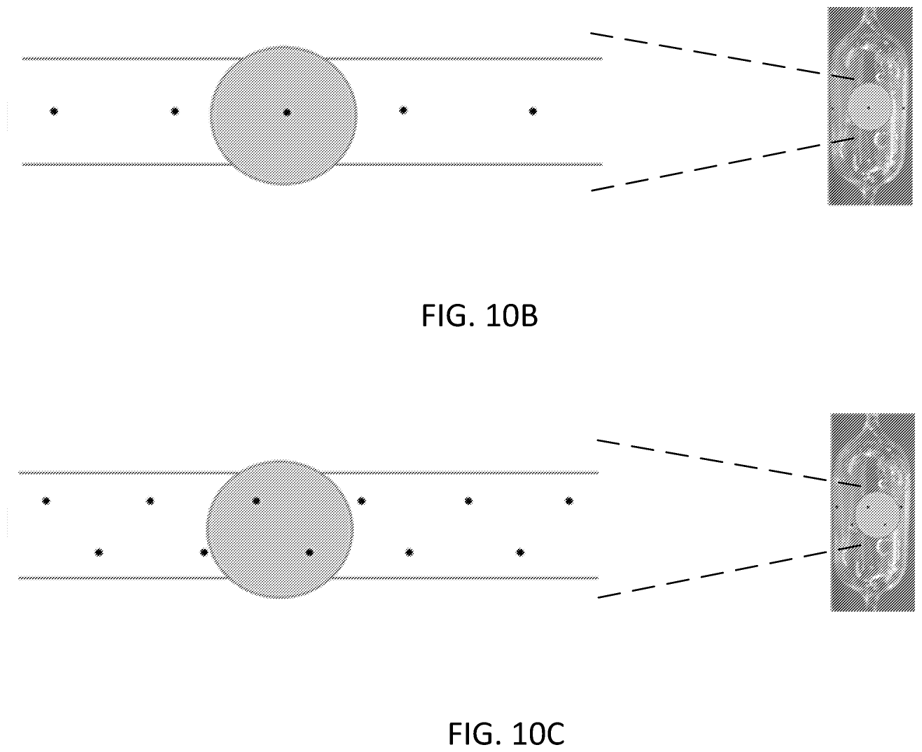

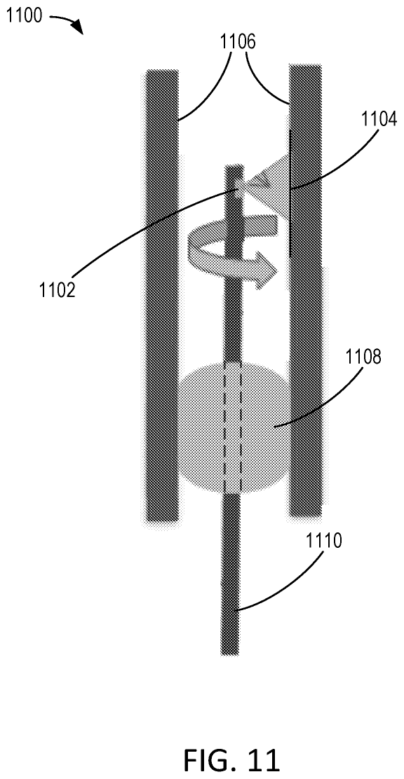

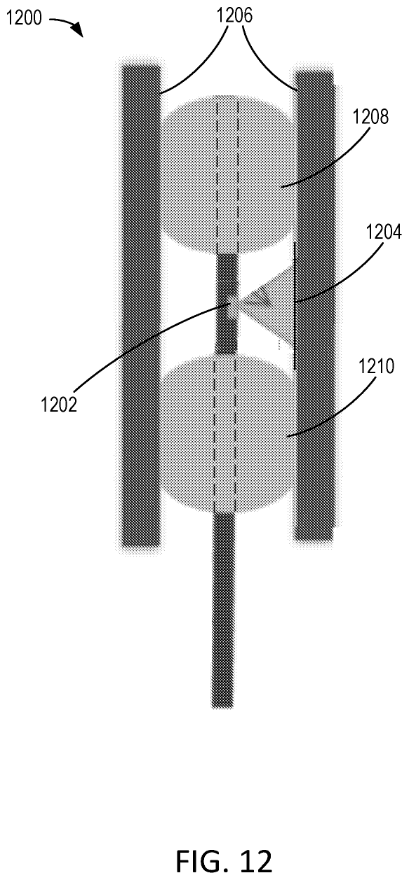

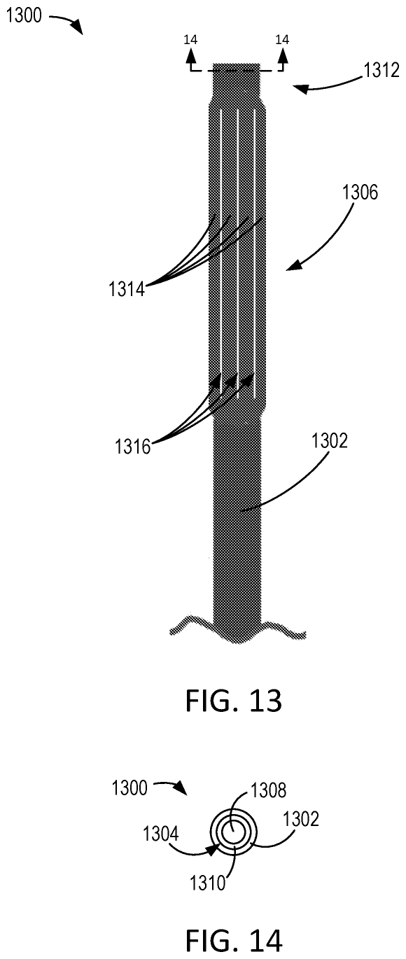

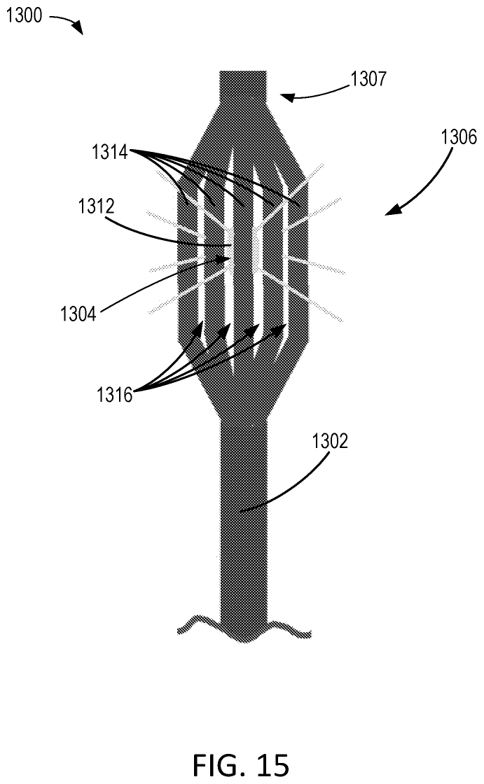

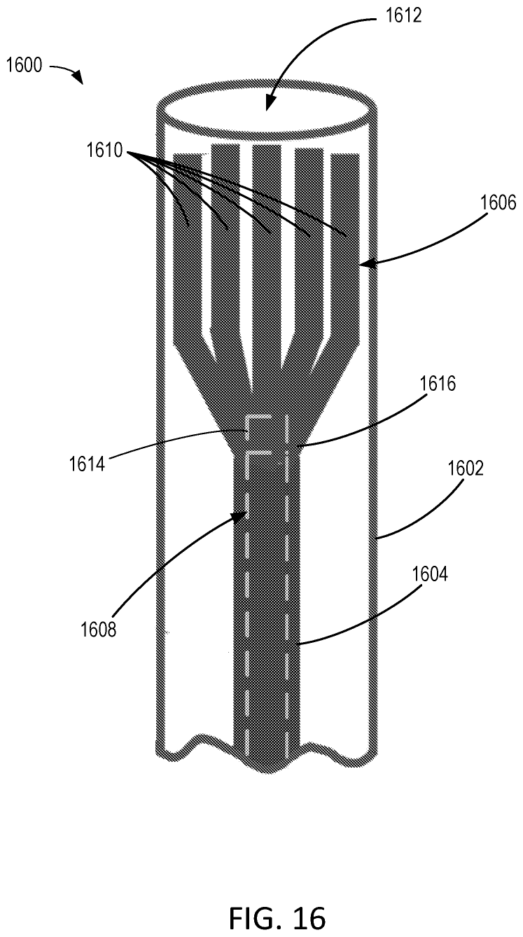

In one non-limiting example, the microholes 148 may be formed in a grid or array, as illustrated in FIG. 10A. However, many other patterns or geometric shapes, both symmetrical and asymmetrical may be utilized, such as will be described. In the non-limiting example provided in FIG. 10A, the microholes 148 may be arranged in a rectangular or square array covering a portion of the expandable device 130 on one radial side of the expandable device 130. For example, the microholes 148 may be arranged in a 1 cm by 1 cm square array. In the illustrated non-limiting example, the expandable device 130 includes 25 microholes 148. However, in other non-limiting examples, there may be between 1 and 100 microholes 148.

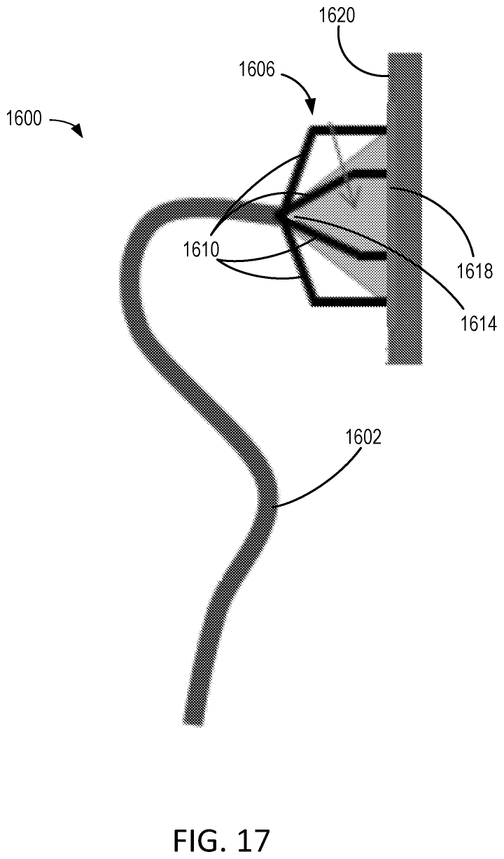

In some other instances, the microholes 148 may be arranged in a single-file line that may extend partially or fully around the circumference of the expandable device 130, as shown in FIG. 10B. In some instances, the microholes 148 may be arranged in multiple rows or single-file lines of microholes 148. In some of these instances, the multiple rows or single-file lines may be offset from each other, such that the microholes 148 are staggered, as shown in FIG. 100.

In some instances, the plurality of microholes 148 may be sized such that they are configured to deliver the photosensitizer therethrough onto the lesion 145 when the inflatable balloon is inflated. In some instances, each microhole 148 of the plurality of microholes 148 may have a diameter between approximately 25 microns and 300 microns. For example, in some instances, each microhole 148 of the plurality of microholes 148 may have a diameter of 75 microns, 100 microns, 125 microns, or 150 microns, as desired for a given photosensitizer. Accordingly, with the expandable device 130 in the expanded position, the photosensitizer may be pushed through the sheath 128, into the expandable device 130, and onto the lesion 145.

In some instances, when the secondary expandable device 152 is provided within the expandable device 130, only the secondary expandable device 152 may be filled with air or fluid, which may then expand and provide structural integrity to the expandable device 130 in the expanded position. The photosensitizer may then be applied into the expandable device 130, outside of the secondary expandable device 152, and may then be infused through or allowed to weep out of the plurality of microholes 148 onto the lesion 145. In these instances, the sheath 128 may have multiple channels to allow for varying fluids to be delivered to the expandable device 130 and the secondary expandable device 152.

In some instances, it may be desired that the photosensitizer be applied only to the lesion 145, and not to the surrounding area of the internal passageway 144 of the subject. Accordingly, the expandable device 130 may include a rotational marker or sight bead 147 (shown in FIG. 4) to aid in the rotational alignment of the expandable device 130 within the internal passageway 144. As such, the catheter 118 may be rotatable within the sheath 128 to allow for rotational alignment by the healthcare professional during treatment. In some instances, the catheter 118 may be aligned by manually rotating the catheter 118. In some other instances, the endoscopic device 114 may be equipped with a selectively rotatable knob configured to precisely rotate the catheter 118 about its longitudinal axis.

With the photosensitizer applied to the lesion 145, the light source 132 may be advanced through the sheath 128, at step 816. The light source 132 may be advanced until the light source 132 is disposed within the expandable device 130, proximate the lesion 145. Similar to the advancement indicators 146, the light source 132 may include an advancement marker 150 (shown in FIG. 6). The advancement marker 150 may allow for the accurate placement of the light source 132 within the expandable device 130 during use. Specifically, the advancement marker 150 may similarly display a measure of placement of the light source 132 within the expandable device 130. Additionally or alternatively, a guide light (not shown) may be used to ensure accurate placement of the light source 132.

Alternatively, in some instances, when the light source 132 and the sheath 128 both extend entirely through each of the expandable device 130 and the secondary expandable device 152 and are coupled together at a distal end, the light source 132 may be disposed proximate the lesion 145 based on the positioning of the catheter 118, discussed above.

With the light source 132 disposed within the expandable device 130, proximate the lesion 145, the light source 132 may then be used to apply light to the photosensitizer, thereby treating the lesion 145. In some instances, while treating the lesion 145, the light source 132 may be moved along the axial direction to aid in effectively treating the entire lesion 145. In the instances where the photosensitizer comprises Rose Bengal, the light applied by the light source 132 may be green light. In other instances, the light may be a variety of other types of light including, for example, visible light, such as within a range of 450-750 nm or blue light and riboflavin.

Because the light source 132 is either fed through the sheath 128 or coupled to the sheath 128, which is centered within the expandable device 130, the distance from the light source 132 to the lesion 145 may be accurately determined. In some instances, the distance from the light source 132 to the lesion 145 during treatment may correspond to approximately half of the diameter or width of the expanded expandable device 130. This predetermined distance between the light source 132 and the lesion 145 may be used to determine an appropriate power setting for the light source 132 to deliver a desired amount of energy to the photosensitizer, and thereby to the lesion 145 for treatment.

Appropriate power settings for the light source 132 may be determined by establishing various baseline power delivery values using the light source 132. For example, baseline power delivery values may be determined from the light source 132 applying light through air (to establish an air baseline), through the balloon or expandable device 130 inflated with air (to understand how much the balloon attenuates the light power compared to the air baseline), and through the balloon or expandable device 130 filled with water (to understand how much the water attenuates the light power). In some instances, these power settings may be determined during a bench testing procedure. Accordingly, the power settings may be determined using a device similar to or identical to the expandable device 130.

In the instances where the expandable device 130 is filled with water, a lipid solution, a mixture of intralipid and water, or another dispersing solution, the water, lipid solution, mixture of intralipid and water, or other dispersing solution may provide cooling to the expandable device 130 to prevent deterioration of the expandable device 130 during treatment.

In some instances, the light from the light source 132 may be directed through a window 149 (shown in dashed lines on FIG. 4) on the expandable device 130 to control a direction of light delivery from the light source 132 to the subject. In these instances, the expandable device 130 may form a mask outside the window 149 comprising a translucent or opaque coating, and the portion of the expandable device 130 disposed within the window 149 may comprise a transparent or near-transparent material. In other instances, the light from the light source 132 may be dispersed a full 360 degrees around the internal passageway 144.

After treating the lesion 145 with the light source 132, each of the expandable device 130 and the secondary expandable device 152 may then be moved into the collapsed position, and the catheter 118 may be removed from the internal passageway 144 by pulling the catheter 118 back through the working channel 124. In the instances where the expandable device 130 and the secondary expandable device 152 are inflatable balloons, the expandable device 130 and/or the secondary expandable device 152 may be moved into the collapsed position by applying a negative pressure to the corresponding inflatable balloon. In some instances, where the expandable device 130 includes the plurality of microholes 148, the secondary expandable device 152 may be coupled to the expandable device 130 at various locations, such that deflation of the secondary expandable device 152 results in the collapsing/deflation of the expandable device 130. For example, the secondary expandable device 152 may be tacked or adhered onto the expandable device 130 at various axial and circumferential locations to allow for the expandable device 130 to collapse with the secondary expandable device 152. In these instances, the negative pressure may only be applied to the secondary expandable device 152.

It will be appreciated that the above described systems, devices, and methods have been provided as non-limiting examples, and that various alterations or substitutions may be made to any of the systems, devices, and methods without departing from the scope of the present disclosure.

For example, in the above-described catheter 118, the light source 132 is configured to apply light to the internal passageway 144 through the expandable device 130, which may be an inflatable balloon. However, in various other catheters, the light source may be configured to apply light to the internal passageway of the subject proximal or distal to the expandable device 130.

For example, referring to now to FIG. 11, a catheter 1100 is provided in which a light source 1102 applies light to a lesion 1104 within an internal passageway 1106 of a subject, distal to an expandable device 1108. The light source 1102 may be enveloped by a guide wire 1110 configured to aid in the accurate placement of the light source 1102 within the internal passageway 1106.

The light source 1102 may similarly comprise a fiber optic cable cut at an angle to direct light through an opening in the guide wire 1110. The light source 1102 and the guide wire 1110 may be configured to pass entirely through the expandable device 1108. The expandable device 1108 may again comprise an inflatable balloon. Accordingly, the expandable device 1108 may similarly be configured to be selectively inflated and deflated between an expanded or inflated position (as illustrated in FIG. 11) and a collapsed or deflated position (similar to that of the expandable device 130 depicted in FIG. 3). The catheter 1100 may similarly be sized such that, when the expandable device 1108 is in the collapsed position, the catheter 1100 may be passed through the working channel 124 of the endoscopic device 114.

Referring now to FIG. 12, a catheter 1200, similar to the catheter 1100, is provided. However, the catheter 1200 alternatively includes a light source 1202 that is configured to apply light to a lesion 1204 within an internal passageway 1206 proximal to a first expandable device 1208.

Further, the catheter 1200 additionally includes a second expandable device 1210, proximal to both the first expandable device 1208 and the light source 1202. The second expandable device 1210 may, in conjunction with the first expandable device 1208, aid in ensuring that the light source 1202 is centered within the internal passageway 1206.

Additionally, in some applications, the photosensitizer may be applied between the first expandable device 1208 and the second expandable device 1210, such that the first and second expandable devices 1208, 1210 collectively prevent the photosensitizer from being applied proximal to the second expandable device 1210 or distal to the first expandable device 1208.

Each of the first and second expandable devices 1208, 1210 may similarly be selectively actuatable between an expanded position and a collapsed position. Accordingly, the catheter 1200 may similarly be sized such that, when both the first and second expandable devices 1208, 1210 are in the collapsed positions, the catheter 1200 may be passed through the working channel 124 of the endoscopic device 114.

Although the expandable devices 130, 1108, 1208, 1210 described above each comprise inflatable balloons, it will be appreciated that various other types of expandable devices may be additionally or alternatively used.

For example, referring now to FIGS. 13-15, a catheter 1300 is provided. The catheter 1300 includes an outer sheath 1302, a light source 1304 (shown in FIG. 14), and an expandable portion 1306. The outer sheath 1302 may envelop the light source 1304 and be coupled to the light source 1304 at a distal end 1307 of the catheter 1300. The light source 1304 may similarly comprise a fiber optic cable 1308 at least partially enveloped within and coupled to a guide wire 1310. The fiber optic cable 1308 may extend into the expandable portion 1306 and include an emission portion 1312 configured to emit light through a radial opening (not shown) in the guide wire 1310. For example, the emission portion 1312 of the fiber optic cable 1308 may present an opening forming at any of a variety of angles, for example, angles of 0-180 relative to an optical path through the fiber optic cable 1308. As illustrated, the expandable portion 1306 may comprise a portion of the outer sheath 1302 having a plurality of struts 1314 separated by a plurality of axially-extending slits 1316.

The expandable portion 1306 may be selectively actuated between a collapsed position (shown in FIG. 13) and an expanded position (shown in FIG. 15). For example, the expandable portion 1306 may be moved from the collapsed position to the expanded position by advancing the outer sheath 1302 distally relative to the light source 1304. Because the outer sheath 1302 and the light source 1304 are coupled together at the distal end 1307 of the catheter 1300, as the outer sheath 1302 is moved distally, the plurality of axially-extending slits 1316 allow for the plurality of struts 1314 to bend radially outward, thereby expanding the expandable portion 1306. Thus, in the expanded position, the light source 1304 may be configured to apply light through the plurality of slits 1316 of the expandable portion 1306.

In some instances, the catheter 1300 may similarly be used within the esophagus of a subject. In these cases, the expandable portion 1306 may have a maximum expanded width or diameter of 20 mm. In some other instances, the expandable portion 1306 may have a maximum expanded width or diameter between 14 mm and 20 mm.

Further, similar to the catheter 118 described above, when the expandable portion 1306 is in the collapsed position, the catheter 1300 may be configured to pass through the working channel 124 of the endoscopic device 114 described above.

Thus, during use, the catheter 1300 may be inserted into an internal passageway of a subject. With the catheter 1300 inserted into the internal passageway of the subject, the expandable portion 1306 may then be moved into the expanded position. As the expandable portion 1306 is moved into the expanded position, outer surfaces of the plurality of struts 1314 may contact an inner surface of the internal passageway, proving an opening force on the internal passageway. Then, with the expandable portion 1306 in the expanded position, the light source 1304 may be configured to apply light between adjacent struts 1314 to a photosensitizer-treated lesion within the internal passageway of a subject, as described above, with reference to the catheter 118.

In some instances, the outer sheath 1302 may be configured to permit a photosensitizer to be passed therethrough. In these instances, when the expandable portion 1306 is in the expanded position, the catheter 1300 may be configured to selectively apply photosensitizer to the lesion within the subject between adjacent struts 1314.

Referring now to FIGS. 16 and 17, a catheter 1600 is provided. The catheter 1600 comprises an outer sheath 1602, an inner sheath 1604, a frame 1606, and a light source 1608. The outer sheath 1602 may be comprised of a selectively actuatable material configured to aid in directing the outer sheath 1602. For example, the outer sheath 1602 may be made of a material that is reactive to (i.e., is configured to bend in a predetermined direction in response to) an applied voltage, such that the catheter 1600 may be directed by selectively applying voltage to the outer sheath 1602. In some instances, the outer sheath 1602 may be comprised of multiple materials, and may include axial strips of the selectively actuatable material, as described above, separated by non-reactive material, such that the catheter 1600 may be selectively directed in multiple directions by selectively applying voltage to each of the various axial strips. In some instances, the catheter 1600 may alternatively or additionally include mechanical pull wires configured to selectively direct the catheter 1600. For example, the catheter 1600 may include an adjustment knob or several adjustment knobs at a proximal end of the catheter 1600 that are configured to selectively pull the mechanical pull wires, which may be attached at different circumferential locations around the circumference of a distal end of the catheter 1600, to selectively direct the catheter 1600.

The inner sheath 1604 may be movable relative to the outer sheath 1602 along an axial direction. The frame 1606 may be coupled to a distal end of the inner sheath 1604. The frame 1606 may include a plurality of arms 1610 configured to expand radially outward when the frame 1606 is moved axially out of a lumen 1612 of the outer sheath 1602 (as shown in FIG. 17). In some instances, the frame 1606 may comprise a shape memory material. For example, in some instances, the frame 1606 may be made of nitinol. In some other instances, the frame 1606 may be made of various other materials, such as, for example, nitinol, stent braids, or the like.

The light source 1608 may be disposed within the inner sheath 1604 and may comprise a fiber optic cable including a light emission point 1614 configured to emit light therefrom. The light source 1608 may be arranged within the inner sheath 1604 such that the light emission point 1614 is disposed proximate a base 1616 of the frame 1606.

As alluded to above, the frame 1606 may be actuated between a compressed position (as shown in FIG. 16) and an expanded position (as shown in FIG. 17). When the frame 1606 is in the compressed position (i.e., disposed within the outer sheath 1602), the catheter 1600 is similarly configured to be passed through the working channel 124 of the endoscopic device 114 described above. When the frame 1606 is in the expanded position (i.e., disposed outside of the outer sheath 1602), a healthcare professional may arrange the frame 1606 onto a lesion 1618 on an internal surface 1620 of a subject (as shown in FIG. 17) by advancing the catheter 1600 through the working channel 124 of the endoscopic device 114 and selectively actuating the selectively actuatable material of the outer sheath 1602 to direct the catheter 1600, as described above.

With the expanded frame 1606 disposed on the lesion 1618, the light emission point 1614 of the light source 1608, which is located at the base 1616 of the frame 1606, may be retained at a predetermined distance away from the lesion 1618, thereby allowing for an accurate dosage of light energy to be applied to the lesion 1618. Similar to the method 800, described above, the lesion 1618 may be pre-covered with a photosensitizer, as described above.

### EXAMPLES

### Example 1

In one, non-limiting study, esophageal lesions were created with interventions done through an endoscope. The study sought to treat the lesions with the above-described dye delivery balloon and laser light delivery balloon delivered through the working channel of the endoscope. A tensiometry was performed on the tissue samples.

In one study, a system such as described above was tested. More particularly, the system included an infusion balloon catheter and a laser fiber delivery balloon catheter. The infusion balloon catheter included a custom CRE balloon (standard CRE balloon 20-mm nominal extrusion with custom molds to 20-mm length). Balloons were femto laser drilled to include 0.002″ diameter holes arranged in two columns to include 13 holes in total. Two circumferential columns of holes may be desirable in at least some applications. The balloons were UV adhesively bonded to the catheter shaft. However, it was contemplated that the balloon could be laser welded to the catheter, which would reduce the balloon profile by about 0.010′ and improve tracking through the endoscope.

A balloon was wrapped for the cryo-balloon process and the balloons were swabbed with a silicone-based lubricant, in this non-limiting example, MDX. A balloon protector was placed over the balloon to retain wrapped profile (ID of balloon protector approximately 0.100″). Particularly, the balloons were folded, heat set, and wrapped. A tighter wrap could be achieved, which would further contribute to reduced balloon profile and ease of delivery. An improved wrapping process would also improve the rewrap of the balloon after it is inflated, thus allowing for improved ease of retraction and withdrawal through the endoscope. Improved wrapping might also contribute to less entrapped air within the balloon when inflated with intralipid, thus resulting in improved laser light delivery.

The system delivered through endoscope using a CRE packaging wire inserted into the balloon to improve balloon column strength. The wire was retracted and touhey tightened to seal off wire lumen. Rose Bengal was delivered with a syringe

The laser fiber delivery balloon catheter was formed similarly to that of the infusion balloon, but lacked the infusion holes.

In a first tissue set, esophageal lesions were created manually on swine esophagus/stomach sets. Treatment began at the most distal lesion. The dye delivery balloon (0.002 uM holes) was placed in the working channel of the endoscope using a wire as a guide. The balloon was advanced until it was centered on the target lesion. The guide wire was removed/pulled back. Rose Bengal was then injected through the catheter to inflate the balloon. The dye delivery coated the lesion quickly. The first lesion was on the bottom of the esophagus. After the dye delivery was complete (based on visual inspection), the dye delivery balloon was deflated/collapsed and the balloon was removed by pulling it through the endoscope. The balloon was easily removed. The lesion treated with Rose Bengal was rinsed/flushed using the endoscope. The laser fiber delivery balloon was then placed in the working channel and advanced until it was near the target lesion. A syringe was used to withdraw air from the collapsed balloon, to prevent presence of air bubbles. Intralipid solution (0.5%) was injected to inflate the balloon. Once inflated, the solution was withdrawn and re-injected. A 300 uM fiber was placed into the inflated balloon and placement was guided by the red test laser light. The fiber end was placed so that it was a few millimeters beyond the end of the endoscope. Treatment was done with 5 W input for 4 minutes. After treatment, the fiber was removed and the balloon was deflated by removal of the intralipid. Visual inspection shows that the lesion looked photobleached.

Treatment of the second lesion was completed in the same manner as the first lesion. The second lesion was on the top of the esophagus. Dye delivery was achieved in a similar fashion. The laser fiber delivery balloon was then placed in the working channel and advanced until it was near the target lesion. After a treatment of 5 W input (power meter reading was 200 mW) for 4 minutes was completed, it was noted that the lesion did not appear photobleached. Inspection revealed that the laser fiber has failed.

The balloon used to deliver the laser fiber to the 2nd lesion was the same balloon used for the 1st lesion. The initial attempt to deliver this balloon was done wihtout a wire in the balloon for support. It was difficult to advance this device, so the balloon was removed and a guide wire was placed into the balloon. The balloon was then advanced through the endoscope and into position. The guide wire was removed and replaced with the optical fiber. When the catheter was removed after treatment, it was noted that the catheter was kinked in a location that corresponded to the depth the catheter was advanced without a guide wire. It is possible that during the initial attempt to deliver this balloon without a wire, the catheter shaft was inadvertently kinked and this kink may have contributed to the laser fiber failure.

It was determined that the lesion was not treated. The lesion did not look photobleached. Treatment was completed with a new fiber delivery balloon and a new laser fiber. Treatment was done with 5 W input for 4 minutes. After treatment, the fiber was removed and the balloon was deflated by removal of the intralipid. The lesion looked photobleached.

After the treatments were completed, control lesions were made in the esophagus, with one being between the two treated lesions and one being distal to the treated lesions. Control lesions were not treated with dye or laser light. The lesions were then cut out of the esophagus and used for tensiometry testing.

In a second tissue set, a second swine esophagus/stomach set was set up for treatment. Treatments were completed in same manner as above for the first set. The dye delivery and the laser treatments went smoothly. For one of the treated lesions, dye was delivered twice because it appeared that the lesion was not well stained after the first application.

During tensiometry testing, three treated lesions and three control lesions were prepared and tested on the tensiometer.

As illustrated in Table 1 below, the treated lesions had a higher modulus of elasticity than the untreated lesions.

TABLE 1

| | Control | Treated |
|---|---|---|
| | 1876 | 3205 |
| | 1961 | 3093 |
| | 1312 | 2416 |
| Mean | 1716.3 | 2904.7 |
| Stdev | 352.7 | 426.9 |

As can be seen from Table 1, treated lesions had 69.2% higher modulus of elasticity than the untreated lesions. The difference in the modulus of elasticity was also statistically significant p=0.02 (2 tailed T-test)

Thus, treatment of the lesions resulted in a significant increase in tensile strength of the tissue relative to the control-untreated lesions. Grossly, the treated lesions did appear to be photo bleached.

### Example 2

In another, non-limiting study, esophageal lesion were created with interventions done through the endoscope using a snare and ERBE. A 360 degree lesion was targeted around the circumference of the esophagus to determine if dye application and laser light treatment can be delivered in a circumferential manner.

In particular, one male swine was anesthetized and placed on its side for the procedures. The endoscope was inserted into the esophagus. The stomach was at 60 cm. Epiglottis was at 20 cm. A location at 40 cm was selected to make the first lesion. Saline was first in the submucosal space with the interject needle. A bleb was produced, but it was gone by the time the interject needle was removed from the endoscope and the snare was placed through the endoscope and placed near the injection site. A lesion was created using the snare and the ERBE. Visual inspection was performed endoscopically to confirm that the lesion was close to a 360 degree circumference lesion. There were tissue flaps around the lesion that made it difficult to determine the exact size of the lesion. Additional tools, such as suction and graspers could be used to make the lesion.

Treatment was completed using the same methods used in the bench-top study described above as Example 1. Rose Bengal was applied to the lesion with the dye delivery balloon (0.002 uM holes) described above. The balloon was inflated and dye was delivered. The balloon appeared to be well apposed against the esophagus wall. The balloon was removed and the lesion looked well stained with dye, such that the dye appeared to cover the 360 circumference of the lesion in visual inspection.

The laser light delivery balloon was inserted into the working channel of the endoscope and advanced to the lesion. Air was withdrawn to create a vacuum. A 0.5% solution of intralipid was injected into the balloon until it was inflated. This balloon also appeared to be well apposed against the esophagus wall. A 300 uM fiber was placed into the inflated balloon and placement was guided by the red test laser light. The fiber end was placed so that it was a few millimeters beyond the end of the endoscope. Treatment was done with 5 W input for 4 minutes. After treatment, the fiber was removed and the balloon was deflated by removal of the intralipid. The lesion appeared photobleached in visual inspection.

The endoscope was moved to ˜32 cm to create a control lesion using the same methods that were used to create the treatment lesion. A ˜360 degree lesion was created using the snare and ERBE. The animal was euthanized and the esophagus was collected.

The treated lesion and the control lesion were prepared and tested on the tensiometer. Several samples were created from treated and control circumferential lesions and the results of the testing modulus of elasticity are provided in Table 2.

TABLE 2

| | Control | Treated |
|---|---|---|
| | 550 | 1508 |
| | 435 | 1034 |
| | 462 | 980 |
| | 590 | 909 |
| Mean | 509.3 | 1107.8 |
| Stdev | 72.9 | 271.7 |

The treated lesions had about a 117.5% higher modulus of elasticity than the untreated lesions. The difference in the modulus of elasticity was statistically significant p=0.005 (2 tailed T-test). Thus, treatment of the lesion resulted in a significant increase in tensile strength of the tissue relative to the control-untreated-lesion. Grossly, the treated lesions did appear to be photobleached. The increase in tensile strength was greater in the in vivo study than the ex vivo study (117% vs 69%). Also, the magnitude of the modulus of elasticity was much greater for the ex vivo study than the in vivo study, however, it is possible that some of this difference may be attributable to the difference in tissue that was tested (namely, previously frozen tissue vs. in vivo/fresh tissue).

The present invention has been described in terms of one or more preferred embodiments, and it should be appreciated that many equivalents, alternatives, variations, and modifications, aside from those expressly stated, are possible and within the scope of the invention.

1. An endoscopic device for treating a lesion within a subject, the endoscopic device comprising:
an endoscope configured to be inserted into the subject, the endoscope including a working channel; and
a catheter configured to pass through the working channel of the endoscope, the catheter comprising:
a sheath having a lumen;
an inflatable balloon configured to be inflated through the lumen of the sheath; and
a light source controllable through the lumen of the sheath to selectively apply light to the lesion within the subject when the endoscope is inserted into the subject.

2. The endoscopic device of claim 1, wherein the inflatable balloon is configured to inflate to position the light source a predetermined distance from an inner surface of the inflatable balloon when the light source is passed through the lumen of the sheath, into the inflatable balloon.

3. The endoscopic device of claim 1, wherein the inflatable balloon includes a plurality of microholes arranged along a portion of the inflatable balloon configured to engage the subject when the inflatable balloon is inflated, wherein the microholes are sized to deliver a photosensitizer to the subject when the inflatable balloon is inflated while the endoscope is inserted into the subject.

4. The endoscopic device of claim 1, wherein the light source includes one of a fiber optic cable extending through the lumen to emit light within the inflatable balloon or a light emitting diode (LED) arranged in the inflatable balloon.

5. The endoscopic device of claim 4, wherein the fiber optic cable includes a distal end presenting an opening forming an angle of 90 degrees or 180 degrees relative to an optical path through the fiber optic cable.

6. The endoscopic device of claim 1, wherein the catheter further includes advancement indicators on the sheath to display a measure of movement of the catheter through the working channel of the endoscope.

7. The endoscopic device of claim 6, wherein the endoscope further includes a lens, and wherein the advancement indicators form marker bands that are viewable using the lens.

8. The endoscopic device of claim 1, wherein the inflatable balloon is selectively movable between a collapsed position sized to move through the working channel of the endoscope and an expanded position sized to engage the subject after being deployed from the working channel of the endoscope.

9. The endoscopic device of claim 1, wherein the inflatable balloon has a maximum diameter between 14 millimeters and 25 millimeters.

10. The endoscopic device of claim 1, wherein the light source is coupled to a guide wire, and the guide wire is configured to arrange the light source in a desired position within the inflatable balloon.

11. The endoscopic device of claim 1, wherein the light source includes an advancement marker on a proximal end, and wherein the advancement marker displays a measure of placement of the light source within the inflatable balloon.

12. The endoscopic device of claim 1, wherein the inflatable balloon forms a mask to control a direction of light delivery from the light source to the subject.

13. The endoscopic device of claim 1, further comprising a scattering medium arranged in the inflatable balloon to scatter light from the light source.

14. The endoscopic device of claim 13, wherein the scattering medium includes at least one of a water-fat emulsion or titanium dioxide solution.

15. The endoscopic device of claim 1, wherein the catheter further comprises a secondary inflatable balloon disposed within the inflatable balloon and coupled to a distal end of the sheath, and wherein the secondary inflatable balloon and the inflatable balloon are configured to operate in concert to deliver a desired dispersion of light from the light source.

16. A method of treating a lesion within a subject using an endoscopic device including an endoscope and a catheter, the endoscope including a working channel, the catheter being configured to pass through the working channel of the endoscope and comprising an inflatable balloon and a light source, the method comprising:
identifying the lesion within the subject;
applying a photosensitizer to the lesion within the subject;
inserting the endoscope into the subject, such that a distal end of the endoscope is proximate the lesion;
advancing the catheter through the working channel, into the subject, proximate the lesion;
inflating the inflatable balloon through the catheter within the subject;
applying light to the photosensitizer using the light source to treat the lesion.

17. The method of claim 16, further comprising advancing the light source into the inflatable balloon, and wherein inflating the inflatable balloon comprises inflating the inflatable balloon a predetermined amount to locate the light source at a predetermined distance from the lesion when the light source is advanced into the inflatable balloon.

18. The method of claim 16, wherein the photosensitizer is applied to the lesion by applying the photosensitizer to an outer surface of the inflatable balloon and placing the outer surface of the inflatable balloon in contact with the lesion.

19. The method of claim 16, wherein the photosensitizer is applied to the lesion by applying the photosensitizer through a plurality of microholes disposed on the inflatable balloon.

20. The method of claim 16, wherein the photosensitizer is Rose Bengal.

21. The method of claim 20, wherein applying light to the photosensitizer using the light source to treat the lesion further comprises applying green light to the Rose Bengal using the light source to treat the lesion.

22. The method of claim 16, wherein the light source is axially movable, and applying light to the photosensitizer further comprises moving the light source axially to treat the entire lesion.

23. An endoscopic device for treating a lesion within a subject, the endoscopic device comprising:
an endoscope configured to be inserted into the subject, the endoscope including a working channel; and
a catheter configured to pass through the working channel of the endoscope, the catheter comprising:
a sheath having a lumen;
an expandable device configured to move between a collapsed position configured to travel through the lumen and an expanded positon to engage the subject when the catheter is positioned within the subject; and
a light source controllable through lumen of the sheath and extending into the expandable device;
wherein the expandable device is configured to engage the subject in the expanded positon to positon the light source to selectively apply light to the lesion within the subject when the endoscope is inserted into the subject.

24. The endoscopic device of claim 23, wherein the expandable device is a balloon or an expandable portion of the sheath.

25. The endoscopic device of claim 24, wherein the expandable portion includes a plurality of struts separated by a plurality of axially-extending slits.

26. The endoscopic device of claim 24, wherein the expandable portion is in the expanded position, the expandable portion has a maximum diameter between 14 millimeters and 25 millimeters.

27. The endoscopic device of claim 23, wherein the light source is enveloped within a guide wire.

28. The endoscopic device of claim 23, wherein the expandable device includes an inflatable balloon coupled proximate a distal end of the sheath and configured to be inflated through the lumen of the sheath;

29. The endoscopic device of claim 23, wherein the expandable device forms a balloon enveloping a portion of the sheath and selectively movable between a collapsed position sized to move through the working channel and an expanded position sized to engage the subject after being deployed from the working channel.

30. The endoscopic device of claim 23, wherein the light source is configured to selectively apply light to the lesion within the subject and distal to the inflatable balloon.

31. The endoscopic device of claim 23, wherein the light source is configured to selectively apply light to the lesion within the subject, proximal to the expandable device.

32. The endoscopic device of claim 23, wherein the expandable device includes a plurality of openings arranged against the subject when in the expanded position and, wherein the openings are sized to deliver a photosensitizer to the subject.
